(19) United States
(12) Patent Application Publication
(10) Pub. No.: US 2013/0028796 A1
(43) Pub. Date: Jan. 31, 2013

(54) PURIFICATION UNIT AND DEODORIDING DEVICE

(71) Applicants: Morio Nakatani, Ichinomiya-shi (JP); Katsutoshi Hibino, Kaizu-shi (JP); Yoshiyuki Matsumura, Yao-shi (JP)

(72) Inventors: Morio Nakatani, Ichinomiya-shi (JP); Katsutoshi Hibino, Kaizu-shi (JP); Yoshiyuki Matsumura, Yao-shi (JP)

(73) Assignee: SANYO Electric Co., Ltd., Moriguchi-shi (JP)

(21) Appl. No.: 13/558,841

(22) Filed: Jul. 26, 2012

(30) Foreign Application Priority Data
Jul. 29, 2011 (JP) 2011-167544

(51) Int. Cl. A61L 9/18

(52) U.S. Cl. 422/121

(57) ABSTRACT

A purification unit for purifying air by a photocatalytic reaction includes a first light source which emits light; a first reflection plate which reflects the light emitted from the first light source; and a plurality of purification plates which cause the photocatalytic reaction by irradiation of the light emitted from the first light source. In this arrangement, at least one of the purification plates is disposed between the first reflection plate and the first light source, and the light emitted from the first light source is transmitted through the purification plate disposed between the first reflection plate and the first light source, and then is reflected on the first reflection plate for irradiation onto the purification plates.

This application claims priority under 35 U.S.C. Section 119 of Japanese Patent Application No. 2011-167544 filed Jul. 29, 2011, entitled “PURIFICATION UNIT AND DEODORIDING DEVICE”. The disclosure of the above application is incorporated herein by reference.

### BACKGROUND OF THE INVENTION

1. Field of the Invention

The present invention relates to a purification unit for purifying a material to be purified that is contained in the air, with use of a photocatalytic structural member, and a deodorizing device.

2. Disclosure of Related Art

In recent years, development has progressed on photocatalytic devices which perform air purification, deodorization, water purification, antibacterial treatment, soil release, water decomposition, with use of a photocatalytic structural member containing a photocatalytically active material. A photocatalytic structural member has such a property that irradiation of light of a predetermined wavelength causes an oxidation-reduction reaction (photocatalytic reaction) on a film surface for purifying a material adhered to the film surface. Generally, the photocatalytic structural member of this kind is formed by laminating a photocatalyst film composed of titanium oxide (TiO₂) or the like on a substrate.

In a purification unit and a deodorizing device incorporated with such a photocatalytic structural member, the air in the vicinity of the device is drawn in through an air intake port, and light to be emitted from a light source is irradiated onto a photocatalyst film for purifying a material to be purified that is contained in the drawn-in air on the photocatalyst film. There is a demand for a technology of guiding the light emitted from the light source to the photocatalyst film in performing the above operation, for efficiently causing the photocatalytic reaction on the photocatalyst film.

### SUMMARY OF THE INVENTION

A first aspect according to the invention is directed to a purification unit for purifying air by a photocatalytic reaction. The purification unit according to the first aspect includes a first light source which emits light, a first reflection plate which reflects the light emitted from the first light source, and a plurality of purification plates which cause the photocatalytic reaction by irradiation of the light emitted from the first light source. In this arrangement, at least one of the purification plates is disposed between the first reflection plate and the first light source. The light emitted from the first light source is transmitted through the purification plate disposed between the first reflection plate and the first light source, and then is reflected on the first reflection plate for irradiation onto the purification plates.

A second aspect according to the invention is directed to a deodorizing device. The deodorizing device according to the second aspect includes the purification unit according to the first aspect, a fan which causes the air to flow into the deodorizing device, and a controller which controls the fan and the purification unit.

### BRIEF DESCRIPTION OF THE DRAWINGS

These and other objects, and novel features of the present invention will become more apparent upon reading the following detailed description of the embodiment along with the accompanying drawings.

FIGS. 1A through 1C are diagrams for describing an arrangement example of a purification plate for causing a photocatalytic reaction by irradiation of light, and FIG. 1D is a diagram showing an arrangement of a purification plate in an example.

FIG. 2 is a diagram for describing an arrangement example of a purification plate for causing a photocatalytic reaction by irradiation of light.

FIG. 3A is a schematic diagram showing a layout of purification plates and a light source in a comparative example, and FIG. 3B is a schematic diagram showing a layout of purification plates and a light source according to a layout principle in the example.

FIG. 4 is an exploded perspective view of a purification unit in the example.

FIG. 5 is a perspective view showing an arrangement of purification plates in the purification unit in the example.

FIG. 6 is a cross-sectional view of the purification unit in the example.

FIG. 7A is a diagram showing reflection which occurs on an interface between purification plates in the example, and

FIG. 7B is a graph showing a simulation result regarding a ratio (reflectance) of reflected light.

FIG. 8 is a diagram showing streams of air in the purification unit in the example.

FIG. 9A is a schematic diagram showing the positions of light sources and reflection plates in pattern 1, and FIGS. 9B through 9E show simulation results regarding a light intensity distribution on a surface of each of the purification plates in pattern 1.

FIG. 10A is a schematic diagram showing the positions of light sources and a reflection plate in pattern 2, and FIGS. 10B through 10E show simulation results regarding a light intensity distribution on a surface of each of the purification plates in pattern 2.

FIG. 11A is a schematic diagram showing the positions of light sources and a reflection plate in pattern 3, and FIGS. 11B through 11E show simulation results regarding a light intensity distribution on a surface of each of the purification plates in pattern 3.

FIG. 12A is a schematic diagram showing the positions of light sources and a reflection plate in pattern 4, and FIGS. 12B through 12E show simulation results regarding a light intensity distribution on a surface of each of the purification plates in pattern 4.

FIG. 13 is a diagram showing an arrangement of a deodorizing device in the example.

The drawings are provided mainly for describing the present invention, and do not limit the scope of the present invention.

### DESCRIPTION OF PREFERRED EMBODIMENTS

In the following, an embodiment of the invention is described referring to the drawings.

<Arrangement Example of Purification Plate>

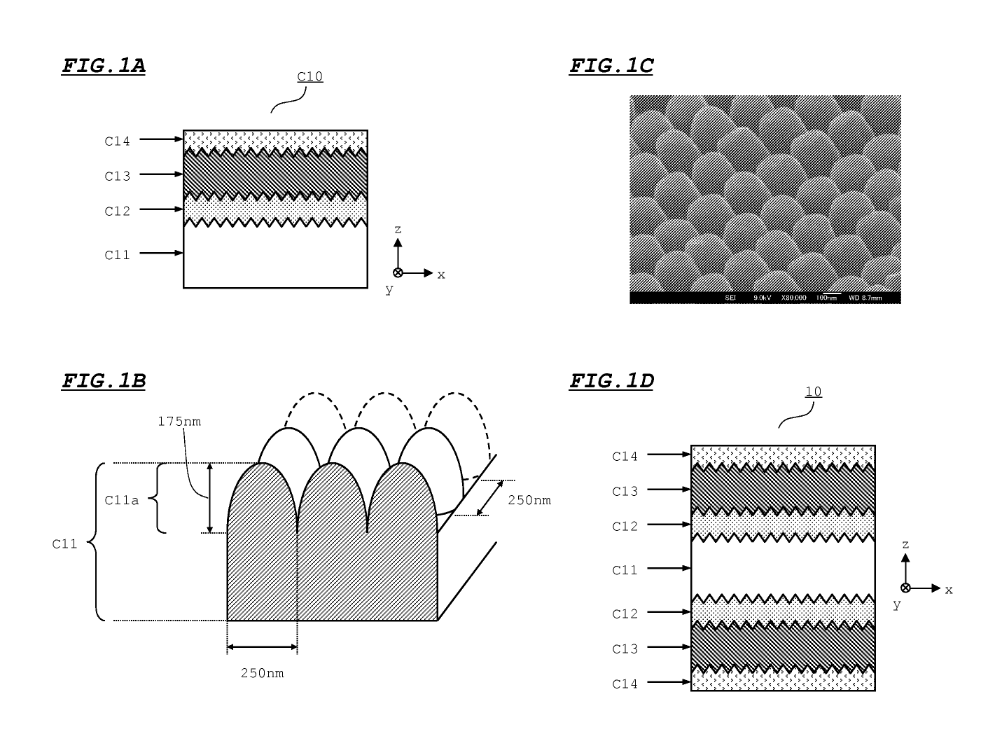

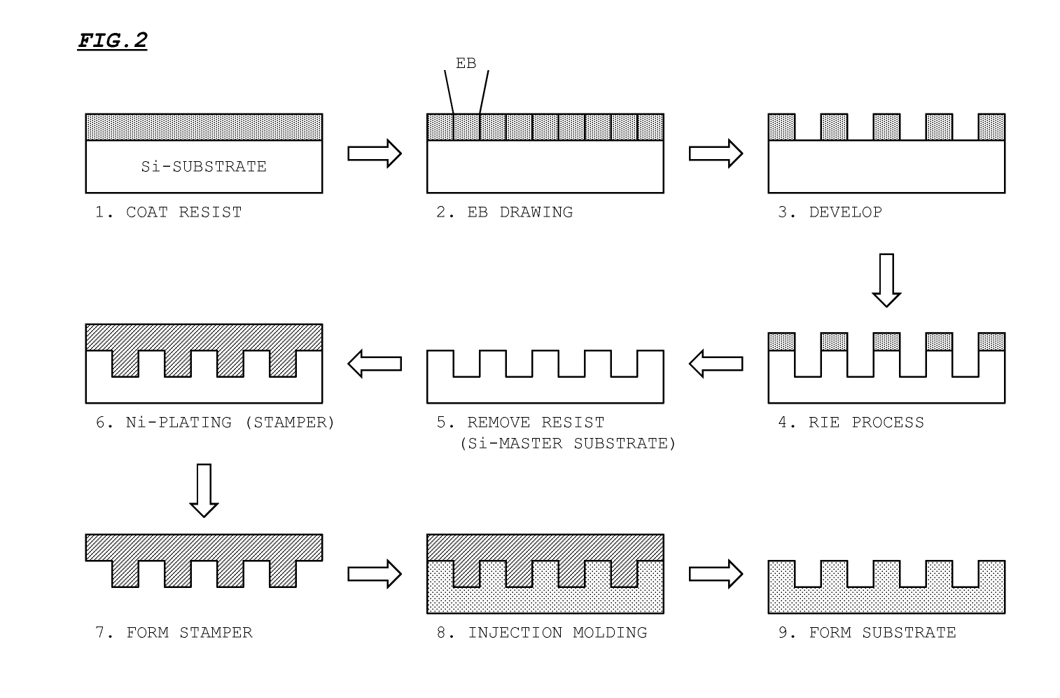

In this section, an arrangement example of a purification plate which causes a photocatalytic reaction by irradiation of light is described referring to FIGS. 1A through 1C and FIG. 2.

FIG. 1A is a diagram showing a laminate structure of a purification plate C10, FIG. 1B is a diagram showing a concave-convex structure C11a of a substrate C11 of the purification plate C10, and FIG. 1C is a diagram showing a secondary electrophotographic image of the concave-convex structure C11a.

Referring to FIG. 1A, the purification plate C10 has the substrate C11, a transparent film C12, a photocatalyst film C13, and an adsorption film C14. The z-axis direction shown in FIG. 1A indicates a lamination direction of the substrate C11, the transparent film C12, the photocatalyst film C13, and the adsorption film C14.

The substrate C11 is made of a light-transparent material such as polycarbonate for transmitting blue light of 405 nm wavelength to be described later at a transmittance of 80% or more, and the refractive index of the substrate C11 is set to 1.6. As shown in FIGS. 1B, 1C, the concave-convex structure C11a is formed on the transparent film C12 side surface of the substrate C11 in such a manner that columnar-shaped protrusions are uniformly arranged at a constant pitch in a matrix. The pitch (width of the columnar-shaped protrusion) of the concave-convex structure C11a is 250 nm in length and breadth, and the height of the columnar-shaped protrusion is 175 nm.

The photographic image shown in FIG. 1C is obtained by forming an alloy film of 20 nm on the concave-convex structure C11a by a sputtering method, followed by image capturing in a state that Pt—Pd is vapor-deposited by 10 Å for electrophotography.

In the following, a sequence of forming the substrate C11 is described referring to FIG. 2.

First, a resist is coated on a silicon master by spin-coating (step 1). Then, a concave-convex structure having the above pitch is formed by EB drawing (electron-beam cutting) (step 2). After the drawing, a developing process is performed (step 3), and then, an RIE process is performed (step 4). Further, the remaining resist is removed by oxygen-plasma asking (step 5). By performing the above steps, a concave-convex structure is formed on the silicon master (Si-master substrate).

Subsequently, the Si master substrate undergoes Ni-plating (step 6) for depositing Ni. Then, a stamper is fabricated by peeling off the deposited Ni-layer from the Si master substrate (step 7). Then, the stamper is subjected to an injection molding process (step 8) for fabricating a substrate C11 (step 9). By performing the above steps, the substrate C11 having a concave-convex structure transferred thereon is formed.

In this embodiment, a light transparent material such as polyolefin may be used as the material for the substrate C11, other than polycarbonate. In particular, it is desirable to use a material having such a property that light absorption on the substrate C11 is small at a use wavelength (light absorption on the substrate C11 is light loss). Preferably, the substrate C11 may not contain impurities or an ultraviolet absorber. Further alternatively, a biodegradable material such as polylactate may be used, other than polyolefin. Use of a biodegradable material is advantageous in reducing e.g. an environmental load, because no harmful substance is generated at the time of disposal, and energy for treatment such as incineration is not required.

Alternatively, laser beam cutting may be applied, in place of EB drawing. In the modification, a photoresist layer is coated on the silicon master. Further, laser light of or about 400 nm wavelength is used as a cutting beam.

Referring back to FIG. 1A, the transparent film C12 is laminated on the concave-convex structure C11a of the substrate C11 formed by the aforementioned sequence by a sputtering method. The transparent film C12 is made of Al₂O₃, and the refractive index of the transparent film C12 is set to 1.6 so that the refractive index of the transparent film C12 is substantially equal to the refractive index of the substrate C11. Further, the upper surface and the lower surface of the transparent film C12 are formed into a concave-convex structure reflecting the concave-convex structure C11a of the substrate C11. Since the transparent film C12 is made of a non-electrolytic inorganic material, the transparent film C12 is free from corrosion by a photocatalytic reaction on the photocatalyst film C13 to be described later. Further, since the refractive index of the transparent film C12 and the refractive index of the substrate C11 are substantially equal to each other, there is an advantage that reflection on interface resulting from a refractive index difference is less likely to occur.

In this embodiment, the film thickness and the Ra (surface roughness) of the transparent film C12 are set to such values that the substrate C11 is not corroded by the photocatalyst film C13. Further, the film thickness and the Ra of the transparent film C12 are set to such values that light to be entered from the substrate C11 side sufficiently impinges the photocatalyst film C13, and that light to be entered from the photocatalyst film C13 sufficiently impinges the substrate C11. The control on the Ra of the transparent film C12 is performed by adjusting the gas pressure at the time of sputtering.

The photocatalyst film C13 is laminated on the upper surface of the transparent film C12 by a sputtering method. The photocatalyst film C13 is made of TiO₂, and the refractive index thereof is set to 2.5. Further, a concave-convex structure reflecting the concave-convex structure formed on the upper surface of the transparent film C12 is formed on the upper surface and the lower surface of the photocatalyst film C13. With this arrangement, a structure reflecting the concave-convex structure C11a on the surface of the substrate C11 is formed on the upper surface (the adsorption film C14 side surface) of the photocatalyst film C13. Thus, the surface area of the upper surface of the photocatalyst film C13 increases, and the number of times of contact between a material to be purified and the photocatalyst film C13 increases, whereby a photocatalytic reaction is likely to occur. Further, since these concave-convex structures have a pitch shorter than the wavelength of light to be irradiated, an apparent refractive index on interface gradually changes, which is advantageous in suppressing reflection. With this arrangement, it is possible to efficiently transmit light in a direction perpendicular to the photocatalyst film C13.

The surface of the photocatalyst film C13 itself after film formation can be made porous by adjusting the gas pressure in laminating. By performing the above operation, the photocatalyst film C13 itself becomes porous, which makes it possible to increase the surface area of the photocatalyst film C13. Further, the surface area of the photocatalyst film C13 can be increased by the concave-convex structure C11a of the substrate C11. If the film thickness of the photocatalyst film C13 is excessively thin, it is impossible or difficult to completely cover the upper surface of the transparent film. C12 by the photocatalyst film C13. On the other hand, if the film thickness of the photocatalyst film C13 is excessively thick, the concave-convex structure formed on the upper surface of the transparent film C12 is not reflected on the upper surface (the adsorption film C14 side surface) of the photocatalyst film C13. In addition to the above, light to be entered from the transparent film C12 side and from the adsorption film C14 side may be absorbed on the photocatalyst film C13, which makes it difficult to transmit the light through the upper surface and the lower surface of the photocatalyst film C13. In view of the above, in this embodiment, the film thickness of the photocatalyst film C13 is set to such a value that the upper surface of the transparent film C12 is sufficiently covered, and that a sufficient amount of light is transmitted through the photocatalyst film C13. In other words, it is desirable to form the film thickness of the photocatalyst film C13 to such a small value as to completely cover the surface of the transparent film C12.

TiO₂ forming the photocatalyst film C13 contains anatase crystal particles. Anatase crystal absorbs ultraviolet light of 388 nm or smaller in wavelength from the band gap energy and causes a photocatalytic reaction according to the band gap thereof. Further, since anatase crystal exists in the photocatalyst film C13 in the form of particles, the anatase crystal is uniformly distributed in the substrate C11, no matter how intricate the shape of the substrate C11 is. This makes it easy to cause a photocatalytic reaction in a wide range over the photocatalyst film C13 with enhanced efficiency.

Further, it is known that TiO₂ forms a rutile structure, an amorphous structure, a brookite structure, other than the anatase crystal structure, and the photocatalytic reaction differs depending on the structure. Specifically, the reaction activity and the reaction wavelength differ in each of the structures. TiO₂ forming the photocatalyst film C13 contains plural types of structures. Specifically, the photocatalyst film C13 composed of TiO₂ has the anatase crystal structure, and is a composite film containing amorphous matter, defects in crystal anatase structure, and particles containing a trace of nitrogen at the time of sputtering, rutile particles. By the containment of these matters, the photocatalytic reaction on the photocatalyst film C13 progresses not only by the light of 388 nm or smaller, which is a reaction wavelength of the aforementioned anatase crystal, but also by the light (blue light) of a wavelength in a visible light region in the range of from 389 to 500 nm. It is preferable to use light to be irradiated onto the photocatalyst film C13 in the wavelength range of from 395 to 410 nm.

The photocatalyst film C13 photocatalytically acts on a material adhered to the photocatalyst film C13. Examples of a material which undergoes a photocatalytic action include ammonium, acetaldehyde, hydrogen sulfide, methyl mercaptan, formaldehyde, acetic acid, toluene, bacteria and oils. These materials undergo a photocatalytic action and are decomposed into carbon dioxide, water, and the like.

The adsorption film C14 is laminated on the upper surface of the photocatalyst film C13 by a sputtering method. The adsorption film C14 is composed of SiO₂ having light transmissivity, and the refractive index thereof is 1.45. SiO₂ has moisture absorbency, and has a property that it is likely to bind with water molecules in the air or a vapor phase gas. With use of the adsorption film C14 having the above property, the material in the air that exists on the upper surface of the adsorption film C14 is easily adhered to the adsorption film C14. Further, the material adsorbed onto the adsorption film C14 is trapped on the adsorption film C14 and is likely to undergo a photocatalytic action.

The adsorption film C14 is laminated on the photocatalyst film C13 in such a manner that the upper surface of the photocatalyst film C13 is not completely coated. Further, if the adsorption film C14 is formed to have such a thickness that reflects the concave-convex structure on the photocatalyst film C13, the refractive index gradually changes, because the concave-convex structure of the adsorption film C14 is shorter than the wavelength of light. With this arrangement, it is less likely to cause reflection on the adsorption film C14, which makes it easy to transmit the light through the adsorption film

C14. Further, forming a concave-convex structure on the photocatalyst film C13 is advantageous in increasing the adsorption rate resulting from an increase in the surface area. In this case, it is more preferable that the adsorption film C14 has a porous structure. Specifically, multitudes of micropores are formed in the adsorption film C14 by lowering the gas pressure at the time of sputtering (specifically, 0.8 through 1 Pa or higher), or increasing the sputtering rate (70 Å/min or larger). By performing the above operation, the material adhered to the upper surface of the adsorption film C14 is contacted with the photocatalyst film C13 through the micropores. Further, the above arrangement allows the light to be entered to the adsorption film C14 to transmit through the adsorption film. C14 so that the light is easily transmitted through the photocatalyst film C13. It is desirable to set the film thickness of the adsorption film C14 to such a value that the material adhered to the adsorption film C14 is efficiently contacted with the photocatalyst film C13 so that light is easily transmitted.

In the case where blue light of 405 nm wavelength is irradiated onto the thus-constructed purification plate C10 from the lower surface of the substrate C11 or from the upper surface of the adsorption film C14, the blue light impinges the photocatalyst film C13. As a result, the material that has entered from the adsorption film C14 side and is in contact with the photocatalyst film C13 is allowed to undergo a photocatalytic action.

In this embodiment, the purification plate which causes a photocatalytic action may be configured to be a purification plate 10 as shown in FIG. 1D. The purification plate 10 is configured in such a manner that a transparent film C12, a photocatalyst film C13 and an adsorption film C14 corresponding to the respective films as shown in FIG. 1A are laminated on the lower surface of the purification plate 10. The thickness of the substrate C11 is set to 0.4 mm, the thickness of the transparent film C12 is set to 7 nm, the thickness of the photocatalyst film C13 is set to 15 nm, and the thickness of the adsorption film C14 is set to 7 nm. With this arrangement, 80% or more of light entered from the upper surface or the lower surface of the purification plate 10 is transmitted through the purification plate 10 and is outputted from a surface opposite to the light entering surface.

With use of the purification plate 10 thus constructed, it is possible to purify the material adhered on the upper surface side and the lower surface side of the purification plate 10. Accordingly, use of the purification plate 10 is advantageous in enhancing the purification performance, as compared with the use of the purification plate C10 shown in FIG. 1A. The transmittance of the purification plate 10 is important. The higher the better, and the transmittance of the purification plate 10 is preferably 80% or more.

In this section, let us consider a case that the purification plates 10 are placed one over the other in a vertical direction, and light is outputted downwardly from an upper side of the uppermost purification plate 10. In this case, if the transmittance is lowered, the light amount at a lower-stage purification plate is exponentially lowered. Specifically, assuming that T is a transmittance of the purification plate 10 and N is the number of purification plates, the ratio (final transmittance ratio) of light to be transmitted downwardly through the lowermost purification plate 10 is expressed by the following formula (1).

$$Final transmittance ratio = {( \frac{T}{100} )}^{N} \qquad ( 1 )$$

It is desirable to design the transmittance of the purification plate 10 so that the value expressed by the formula (1) is 0.1 or more. Then, the light amount of light after transmittance through the lowermost purification plate 10 becomes equal to 10% of the emission light amount. This promotes a minimum photocatalytic reaction on the lowermost purification plate 10. In the formula (1), if T=63%, N=5, the light amount of light after transmittance through the lowermost purification plate 10 is equal to about 10% of the emission light amount. In this case, if the number of the purification plates 10 is larger than five, the photocatalytic reaction efficiency on the lowermost purification plate 10 is extremely lowered. If T=80%, even if N=7, the light amount of light after transmittance through the lowermost purification plate 10 is equal to about 20% of the emission light amount. In this case, a sufficient photocatalytic reaction is obtained even on the lowermost (seventh) purification plate 10. Further, it is possible to increase the number of the purification plates 10 so that the value of the formula (1) becomes equal to or about 0.1.

Next, let us consider a case that the purification plates 10 are placed one over the other in a vertical direction, a reflection plate which reflects light is disposed on an upper side of the uppermost purification plate 10, and light is outputted upwardly from a lower side of the uppermost purification plate 10. In this case, if the number of the purification plates 10 between the reflection plate and a light source is n, the ratio (final transmittance ratio) of light to be transmitted downwardly through the lowermost purification plate 10 is expressed by the following formula (2).

$$Final transmittance ratio = {( \frac{T}{100} )}^{( N + n )} \qquad ( 2 )$$

In this case, it is also preferable to design the number of the purification plates 10, the number of the purification plates 10 between a reflection plate and a light source, and the transmittance so that a minimum photocatalytic reaction is promoted on the lowermost purification plate 10, in other words, so that the value of the formula (2) becomes equal to or larger than 0.1.

In the following example, there are described a purification unit and a deodorizing device incorporated with the purification plate 10.

### <Layout Principle on Purification Plates and Light Sources>

In this section, a layout principle on the purification plates 10, and light sources for irradiating the purification plates 10 with light is described.

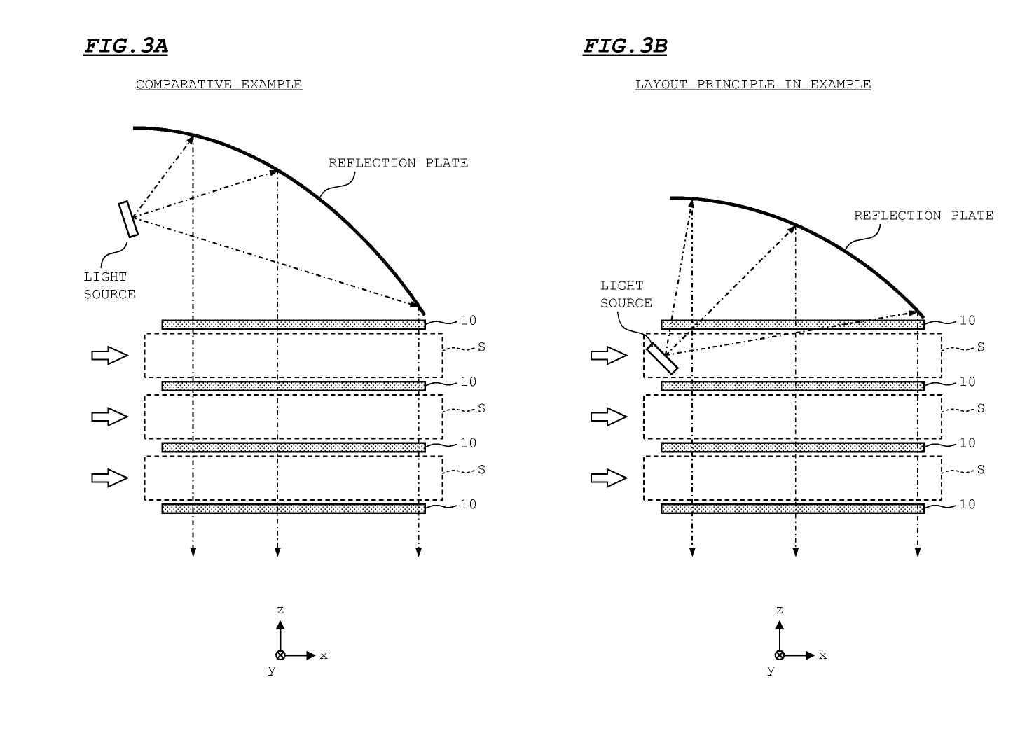

FIG. 3A is a schematic diagram showing a layout (comparative example) of the purification plates 10 and light sources.

In the arrangement shown in FIG. 3A, four purification plates 10 having the same size as each other are provided in a state that the surface of each of the purification plates 10 is disposed perpendicularly to z-axis. The lamination direction of the substrate C11, the transparent film C12, the photocatalyst film C13, and the adsorption film C14 in the purification plate 10 is aligned with z-axis direction, as well as in FIG. 1D. Four purification plates 10 are disposed side by side with a gap S in z-axis direction. The gap S is a space defined between two purification plates 10 adjacent to each other in a vertical direction. More specifically, the gap S is a space defined between a plane including the lower surface of an upper-side purification plate 10 and a plane including the upper surface of a lower-side purification plate 10.

One light source is disposed in x-axis direction, and plural light sources (not shown) are disposed in y-axis direction at an interval in accordance with the y-axis directional width of the purification plate 10. A reflection plate having a curved surface configuration is disposed on the upper side of the uppermost (plus z-axis direction side) purification plate 10. The reflection plate has a shape of a parabolic curve in x-z plane, and the light source is disposed at a focal point on the parabolic curve. The air containing a material to be purified is fed from the left side of the purification plates 10 in a right direction (plus x-axis direction) through the gaps between the adjacent purification plates 10.

In the case where the purification plates 10 and the light source are disposed as described above, light emitted from the light source is entered to each of the four purification plates 10 while transmitting through the purification plates 10. By performing the above operation, the material to be purified which has been contacted with the photocatalyst film C13 of each of the purification plate 10 is purified by a photocatalytic reaction.

In the arrangement shown in FIG. 3A, however, the light reflected on the reflection plate is transmitted downwardly through each of the purification plate 10 only once. Accordingly, the number of times by which the light is transmitted through the purification plates 10 is four times in total, and the light use efficiency is not so high.

FIG. 3B is a schematic diagram showing a layout (a layout principle in the example of the invention) of the purification plates 10 and a light source with enhanced light use efficiency, as compared with the arrangement shown in FIG. 3A.

In the arrangement shown in FIG. 3B, a light source is disposed in a gap defined between the uppermost purification plate 10 and the second uppermost purification plate 10. The reflection plate in the example also has a shape of a parabolic curve in x-z plane, and the light source is disposed at a focal point on the parabolic curve.

In the case where the purification plates 10 and the light source are disposed as described above, light emitted from the light source is entered from the lower surface of the uppermost purification plate 10, is transmitted through the uppermost purification plate 10, and is reflected on the reflection plate. The light reflected on the reflection plate is then transmitted through the uppermost purification plate 10 in minus z-axis direction (downwardly), and is entered to the second uppermost purification plate 10. By performing the above operation, the number of times by which the light is transmitted through the purification plates 10 is five times in total, which is larger than the number of the purification plates 10. Thus, the light use efficiency is enhanced as compared with the arrangement shown in FIG. 3A. This is advantageous in enhancing the purification performance of the four purification plates 10. Further, since the light source is disposed at the focal point on the parabolic curve in x-z plane of the reflection plate, the light reflected on the reflection plate is reflected in a direction perpendicular to the purification plate 10. Thus, the light is allowed to be transmitted through the four purification plates 10 with enhanced efficiency.

Further, in the above arrangement, since the air is fed through the gaps S between the adjacent purification plates 10, and the light source is disposed in one of the gaps S, the light source is cooled by the air flowing through the gap S. This suppresses a temperature rise of the light source, thereby stabilizing the wavelength of light to be emitted from the light source. In particular, in the case where the light source is an LED or a laser, there is a problem that heat may be generated at a light emission point, and the life of the light source may be shortened. However, cooling the light source by the flow-in air enables to extend the life of the light source and stabilize the output of the light source.

In the arrangement shown in FIG. 3B, a light source is disposed in the uppermost gap S among the three gaps S. Alternatively, a light source may be disposed in the other gap S. In the modification, light emitted from the light source is transmitted upwardly through the purification plate disposed between the light source and the reflection plate, and is reflected on the reflection plate. Thus, the modification is also advantageous in enhancing the light use efficiency and in suppressing a temperature rise of the light source, as compared with the arrangement shown in FIG. 3A.

In the following example of a purification unit and a deodorizing device, purification plates and a light source are disposed based on the layout principle shown in FIG. 3B.

### Example of Purification Unit

In the present example, LEDs 21a and 22a correspond to a “first light source” and a “second light source” in the claims. Reflection plates 31 and 32 correspond to a “first reflection plate” and a “second reflection plate” in the claims. A vent 100a corresponds to an “air inlet” in the claims. The description regarding the correspondence between the claims and the present example is merely an example, and the claims are not limited by the description of the present example.

In the following, an example of a purification unit is described.

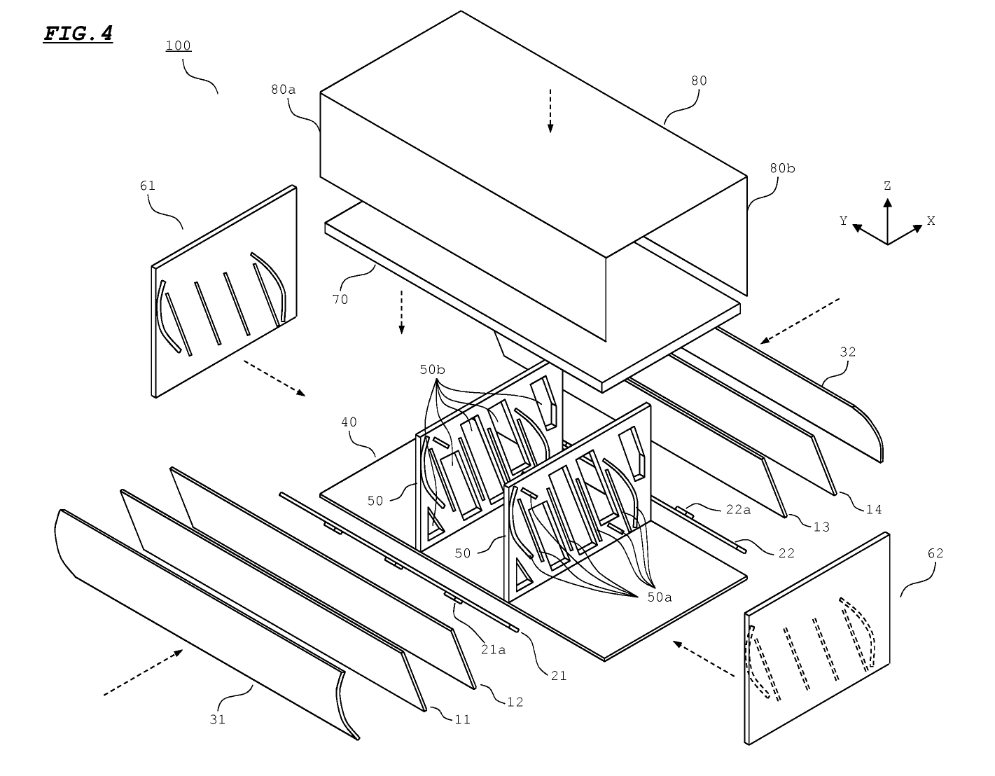

FIG. 4 is an exploded perspective view of a purification unit 100 in the present example.

The purification unit 100 is provided with purification plates 11 through 14, light emitting units 21, 22, reflection plates 31, 32 each having a curved surface configuration, a base member 40, two support plates 50, side plates 61, 62, an upper plate 70, and a cover 80. The purification plates 11 through 14 are configured in the same manner as the purification plate 10 shown in FIG. 1D.

The light emitting units 21, 22 respectively have three LEDs 21a and three LEDs 22a in Y-axis direction. The light emitting units 21, 22 respectively cause the LEDs 21a, 22a to emit light, based on a control signal to be inputted. The LEDs 21a, 22a respectively emit light of 405 nm wavelength toward the reflection plates 31, 32. The directions of the three LEDs 21a disposed in the light emitting unit 21 are the same as each other, and the directions of the three LEDs 22a disposed in the light emitting unit 22 are the same as each other. Three LEDs are disposed in Y-axis direction in each of the light emitting units 21, 22. Alternatively, the number of LEDs to be disposed may be changed, as necessary.

The reflection plates 31, 32 are a mirror having a curved surface configuration. The reflection plate 31 reflects light emitted from the LEDs 21a and transmitted through the purification plate 11 toward the purification plate 11. The reflection plate 32 reflects light emitted from the LEDs 22a and transmitted through the purification plate 14 toward the purification plate 14. The reflection plates 31, 32 have a reflection film for reflecting light of an emission wavelength (405 nm in the present example) from the LEDs 21a, 22a. Specifically, the reflection film is made of alloys such as Ag and Ag+Al, and the reflectance of the reflection film is 80% or more. Further, a higher reflectance is desirable for increasing the light use efficiency.

The support plate 50 is formed with holes 50a for passing through the purification plates 11 through 14, the light emitting units 21, 22, and the reflection plates 31, 32; and with five holes 50b for allowing streams of air to flow in Y-axis direction in regions divided by the support plates 50 within the purification unit 100. The side plate 61 is formed with respective recesses for holding plus Y-axis directional ends of the purification plates 11 through 14 and the reflection plates 31, 32. The side plate 62 is formed with respective recesses for holding minus Y-axis directional ends of the purification plates 11 through 14 and the reflection plates 31, 32. The cover 80 has a top surface in parallel to X-Y plane, and a front portion 80a and a rear portion 80b in parallel to Y-Z plane. The Z-axis directional length of the front portion 80a is shorter than that of the rear portion 80b.

At the time of assembling, the purification plates 11 through 14, the light emitting units 21, 22 and the reflection plates 31, 32 are passed through the holes 50a formed in the two support plates 50. By performing the above operation, the purification plates 11 through 14 are arranged in parallel to each other with a certain gap in the same manner as the four purification plates 10 shown in FIG. 3B. Then, the side plates 61, 62 are set on the base member 40 in such a manner that the ends of the purification plates 11 through 14 and the reflection plates 31, 32 are respectively held in the recesses of the side plates 61, 62. Then, the top plate 70 is set on the upper ends of the two support plates 50, and the side plates 61, 62; and the cover 80 is placed above the top plate 70. In this way, the purification unit 100 is assembled.

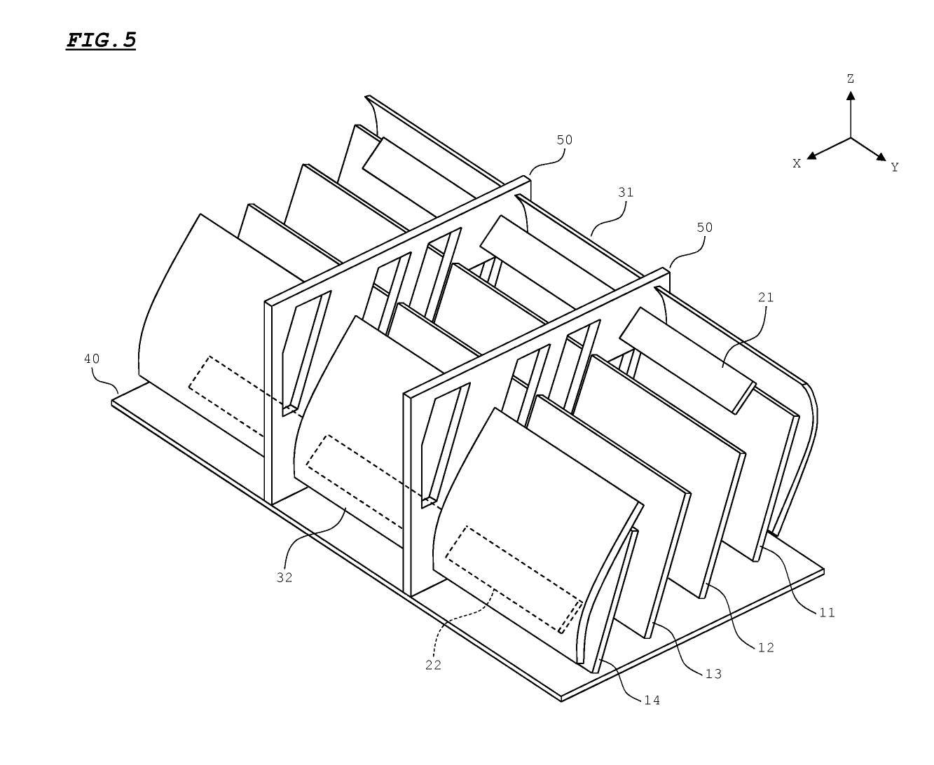

FIG. 5 is a perspective view showing an arrangement of the purification unit 100. To simplify the description, the illustration of the side plates 61, 62, the top plate 70, and the cover 80 is omitted.

The light emitting unit 21 is positioned in the gap defined between the purification plates 11 and 12, and the light emitting unit 22 is positioned in the gap defined between the purification plates 13 and 14.

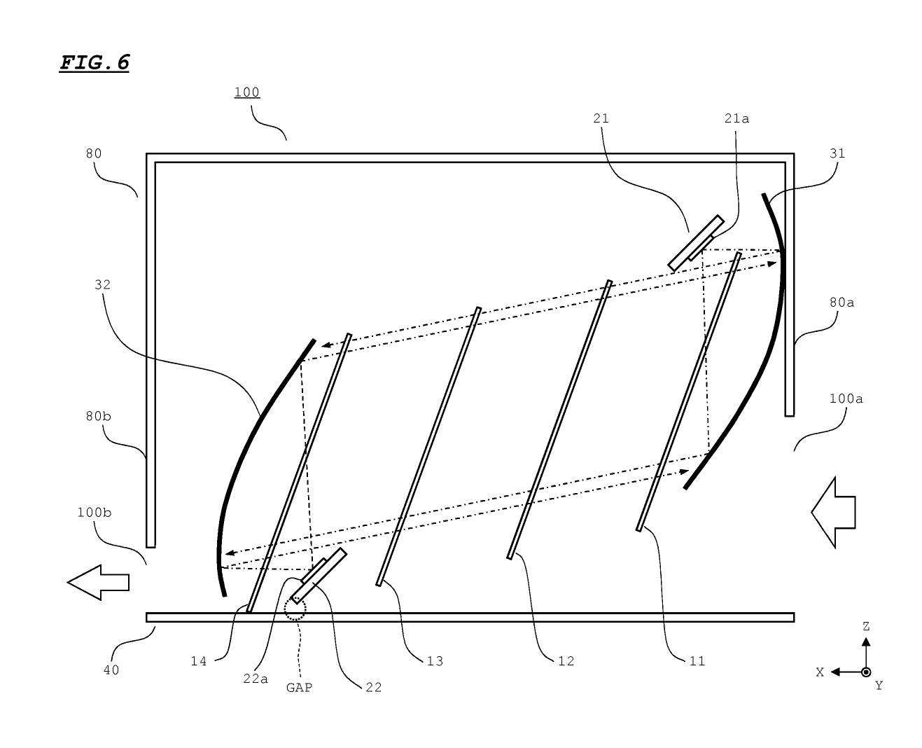

FIG. 6 is a cross-sectional view of the purification unit 100 when viewed in minus Y-axis direction.

The purification plates 11 through 14, the LEDs 21a, and the reflection plate 31 are disposed based on the layout principle shown in FIG. 3B. Specifically, the LEDs 21a are disposed in the gap to be defined between the purification plates 11 and 12. Further, the purification plates 11 through 14, the LEDs 22a, and the reflection plate 32 are also disposed based on the layout principle shown in FIG. 3B. Specifically, the LEDs 22a are disposed in the gap to be defined between the purification plates 13 and 14.

The divergence angle of light to be emitted from the LEDs 21a, 22a is 120 degrees, and the LEDs 21a, 22a respectively irradiate light toward the reflection plates 31, 32. The light emitting units 21, 22 (LEDs 21a, 22a) are disposed with an inclination of 45 degrees with respect to X-axis in X-Z plane. Further, there is defined a gap between the lower end of the light emitting unit 22 and the top surface of the base member 40. The purification plates 11 through 14 are disposed with an inclination of 20 degrees with respect to Z-axis in X-Z plane. Further, the purification plates 11 through 14 are disposed at different positions from each other in Z-axis direction with respect to the base member 40. Specifically, the lower end of the purification plate 14 is grounded to the top surface of the base member 40, and the purification plates 13, 12, 11 are located away from the top surface of the base member 40 in this order.

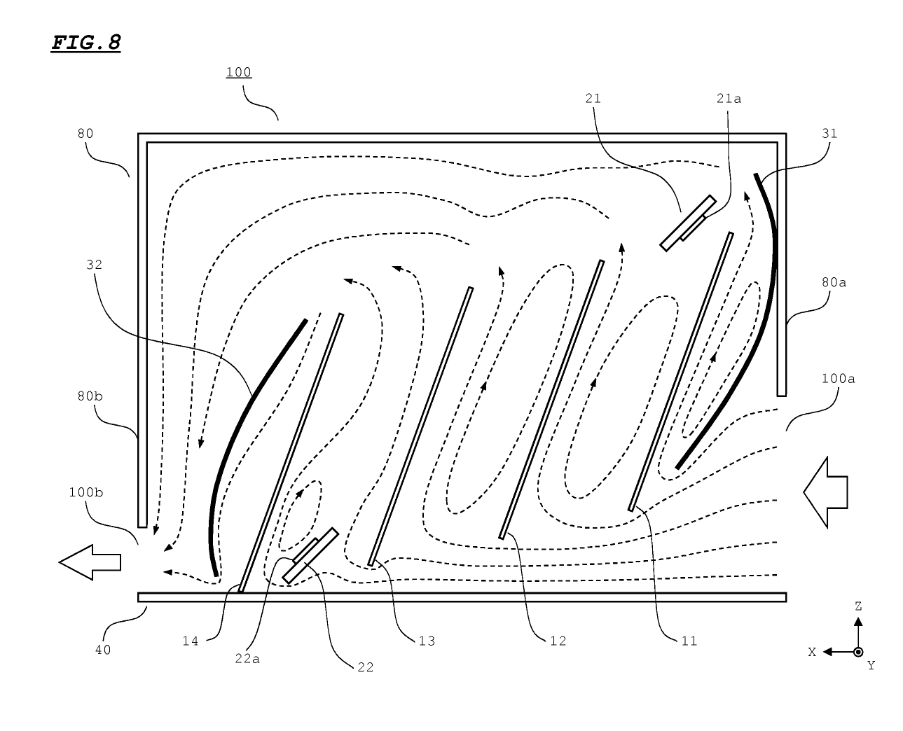

A vent 100a for drawing in the external air into the purification unit 100 is defined between the front portion 80a and the base member 40. Further, a vent 100b for drawing out the air in the purification unit 100 to the outside of the purification unit 100 is defined between the rear portion 80b and the base member 40. The streams of air will be described later referring to FIG. 8.

Light emitted from the LEDs 21a in the light emitting unit 21 is entered to a left surface (plus X-axis direction side surface) of the purification plate 11. The light entered to the left surface of the purification plate 11 is transmitted through the purification plate 11 and is reflected on the reflection plate 31. The light reflected on the reflection plate 31 is entered to a right surface (minus X-axis direction side surface) of the purification plate 11. The light entered to the right surface of the purification plate 11 is transmitted through the purification plate 11, and is transmitted in the order of the purification plates 12, 13, 14.

Light emitted from the LEDs 22a in the light emitting unit 22 is entered to a right surface of the purification plate 14. The light entered to the right surface of the purification plate 14 is transmitted through the purification plate 14 and is reflected on the reflection plate 32. The light reflected on the reflection plate 32 is entered to a left surface of the purification plate 14. The light entered to the left surface of the purification plate 14 is transmitted through the purification plate 14, and is transmitted in the order of the purification plates 13, 12, 11.

The purification unit 100 in the present example is configured in such a manner that light reflected on the reflection plates 31, 32 is entered to the purification plates 11 through 14 in a direction slightly displaced from a vertical direction, unlike the layout principle shown in FIG. 3B. In the case where the purification plates 11 through 14 are disposed with a slight inclination with respect to light to be entered, the ratio (light loss ratio) of light to be reflected on the purification plates 11 through 14 may increase depending on the incident angle of light to be entered to the purification plates 11 through 14. In view of the above, in the present example, the incident angle of light reflected on the reflection plates 31, 32 with respect to the purification plates 11 through 14 is set in such a range as to suppress the light loss ratio.

In the present example, as described above, light can be efficiently used while suppressing reflection as much as possible by the concave-convex structure of each of the layers of the purification plate 10. However, reflection may occur between layers having different refractive indexes, taking into consideration of the refractive index of a material composing each layer.

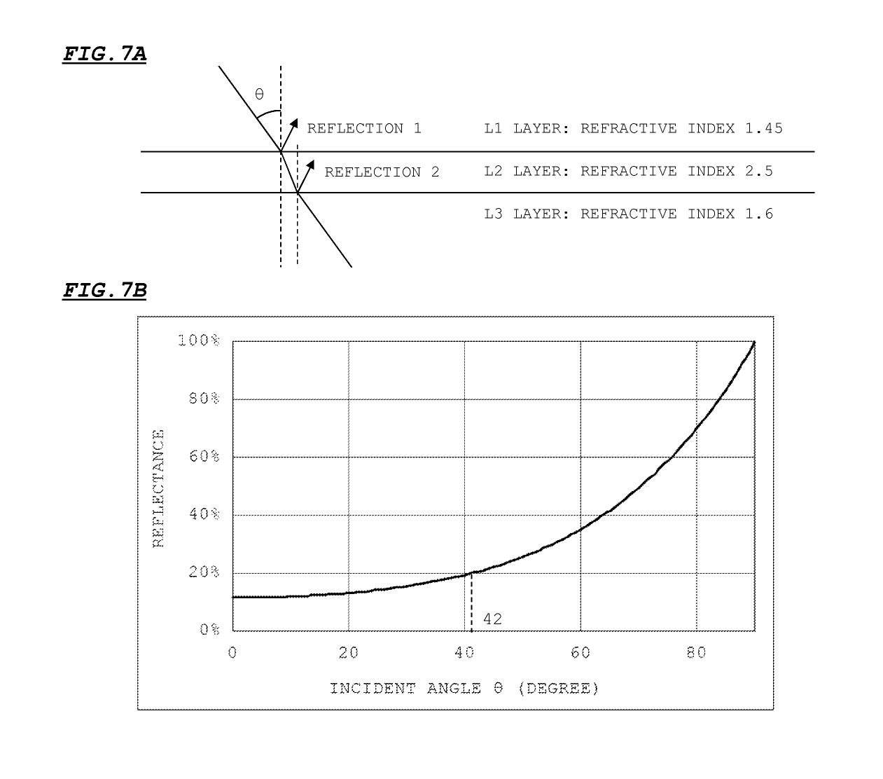

In this example, three layers having different refractive indexes may be formed on one side surface of the purification plate 10. Specifically, let us consider a case that an L1 layer (refractive index: 1.45) corresponding to the adsorption film C14, an L2 layer (refractive index: 2.5) corresponding to the photocatalyst layer C13, and an L3 layer (refractive index: 1.6) corresponding to the transparent film C12 and the substrate C11 are formed. In this case, as shown in FIG. 7A, if light is entered from the L1 layer side with an incident angle θ, reflection 1 occurs at interface between the L1 layer and the L2 layer, and reflection 2 occurs at interface between the L2 layer and the L3 layer.

FIG. 7B is a diagram showing a simulation result indicating a ratio of light (reflectance) as a sum of reflection 1 and reflection 2, with respect to light to be entered from the L1 layer side in the state shown in FIG. 7A. FIG. 7B shows that the reflectance exceeds 20% if the incident angle exceeds 42 degrees. In view of the above, light is required to enter with an angle smaller than 42 degrees. Idealistically, it is preferable to set the incident angle to 0 degree (namely, perpendicular to a light entering plane). If the incident angle is 42 degrees and the reflectance is 20%, the transmittance of the one side surface (L1 layer through L3 layer) becomes 80%. Since the L1 layer through L3 layer are disposed on both sides of the purification plate 10, the ratio of light transmitting through the purification plate 10 becomes 64%. In this case, if light is emitted toward the five purification plates 10, the light amount of light after transmittance through all the purification plates 10 is as small as about 10% of the light amount at the time of light emission. Therefore, if the incident angle exceeds 42 degrees, the light amount of light after transmittance through the fifth purification plate 10 is as small as 10% or less of the light amount at the time of light emission. This extremely lowers the photocatalytic reaction efficiency.

FIG. 8 is a diagram showing streams of air in the purification unit 100. FIG. 8 is a cross-sectional view of the purification unit 100 when viewed in minus Y-axis direction, as well as FIG. 6.

As described above referring to FIG. 6, the members (the purification plates 11 through 14, the light emitting units 21, 22, and the reflection plates 31, 32) in the purification unit 100 are disposed in the purification unit 100. The space within the purification unit 100 surrounded by the base member 40, the support plates 50, the side plates 61, 62, the top plate 70, and the cover 80 is divided by the purification plates 11 through 14, the light emitting units 21, 22, and the reflection plates 31, 32, whereby a channel is formed in the purification unit 100. The external air drawn in through the vent 100a is drawn out through the vent 100b via the channel. In FIG. 8, streams of air that has been drawn in from the outside of the purification unit 100 and drawn out along the channel in the purification unit 100 to the outside of the purification unit 100 are indicated by the broken-line arrows.

The air drawn into the purification unit 100 through the vent 100a is allowed to flow in the space between the purification plates 11 through 14, and the reflection plates 31, 32. When the drawn-in air is allowed to flow, the material to be purified that is contained in the drawn-in air is adhered to the adsorption films C14 of the purification plates 11 through 14. By performing the above operation, as described above, the material to be purified in the air in the vicinity of the purification plates 11 through 14 are stagnated on the adsorption films C14, and are contacted with the photocatalyst films C13. When the light emitted from the LEDs 21a, 22a in the light emitting units 21, 22 is irradiated onto the photocatalyst films C13 in this state, a photocatalytic reaction occurs, and the material to be purified that is in contact with the photocatalyst films C13 is decomposed. The air purified by decomposition of the material to be purified is drawn out through the vent 100b.

Further, as shown in FIG. 8, the purification plates 11 through 14 are disposed at such positions that the distance between the purification plates 11 through 14 and the base member 40 is gradually decreased, as the purification plates 11 through 14 are located away from the vent 100a; and that the lower end of the purification plate 14 is grounded to the top surface of the base member 40. With this arrangement, as shown in FIG. 8, the streams of external air drawn in through the vent 100a are gradually blocked by the purification plates 11 through 14, and are completely blocked by the purification plate 14. Thus, the streams of external air drawn in through the vent 100a are efficiently separated by the gaps to be defined between the purification plates 11 through 14. The distance between the purification plates 11 through 14 and the base member 40 is set to such a value that the streams of external air drawn in through the vent 100a are substantially uniformly distributed in the gaps to be defined between the purification plates 11 through 14.

Further, if the purification plates and the light sources are disposed as described above, it is possible to separate the streams of air drawn into the purification unit 100 from the streams of air (streams of air in plus X-axis direction) immediately before the external air is drawn into the purification unit 100. With this arrangement, even if the X-axis directional width of the purification unit 100 is reduced, it is possible to allow the drawn-in air to flow along the channel for a sufficiently long time, and to extend the distance by which the drawn-in air is allowed to pass. Thus, the number of times of contact between the drawn-in air and the photocatalyst films C13 disposed on the channel increases, which enhances the purification efficiency.

Further, the streams of air from the right side of the light emitting unit 22 are allowed to flow near the lower end of the purification plate 14 through the space between the lower end of the light emitting unit 22 and the top surface of the base member 40. Then, the streams of air near the lower end of the purification plate 14 are allowed to flow upwardly along the right surface of the purification plate 14. By performing the above operation, it is possible to decompose the material to be purified by a photocatalytic reaction even at the lower end of the purification plate 14. As indicated by the broken-line arrows shown in FIG. 8, the streams of air separated by the purification plates 14 are allowed to flow near the purification plates 14, the above arrangement is suitable for purification by a photocatalytic action.

### Simulation on Layout

The inventor of the present application conducted simulations regarding a light intensity on the purification plates 11 through 14, in the case where the purification plates through 14 were disposed as described above in the purification unit 100. In the following simulations, the wavelength of light to be emitted from each of the light source is 365 nm, and the divergence angle of light to be emitted from each of the light source is 120 degrees, as well as the case where the LEDs 21a, 22a are used. Further, in any of the simulations, the three light sources are arranged in Y-axis direction, and the intensity of light to be emitted from each of the light source is the same. Further, in any of the simulations, the sum of intensities of light to be emitted from the three light sources arranged in Y-axis direction is the same (0.33×3 watts).

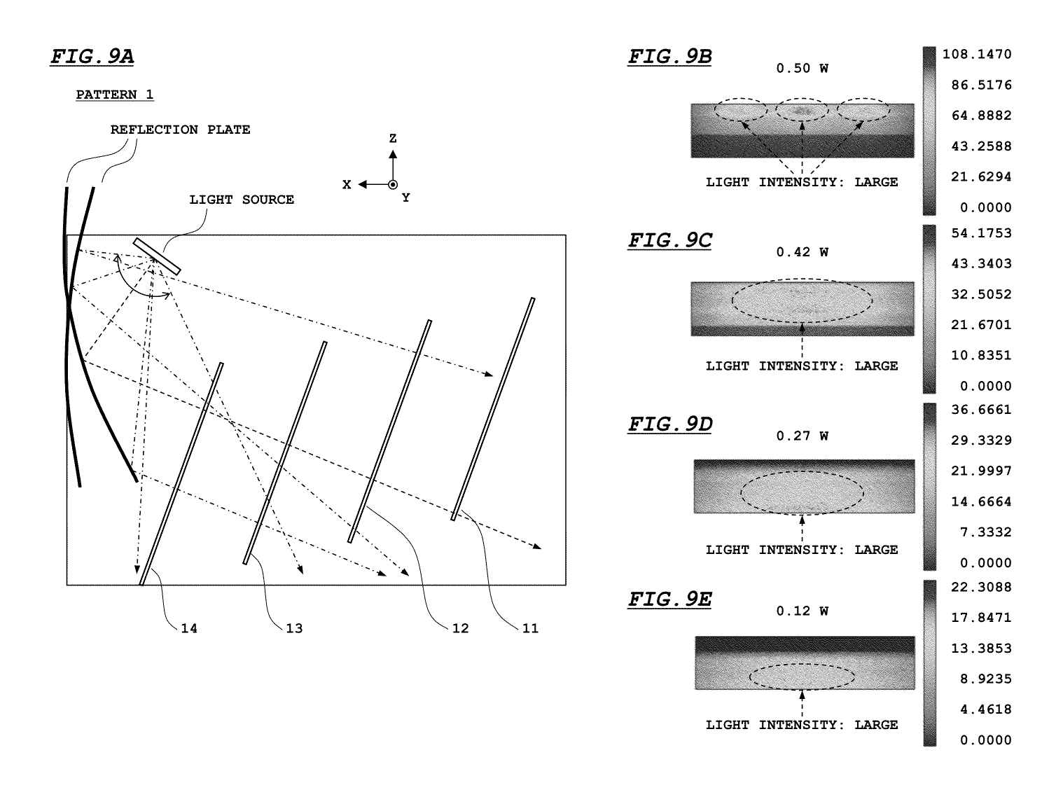

FIG. 9A is a schematic diagram showing the positions of light sources and reflection plates in pattern 1. FIGS. 9B through 9E are simulation results respectively showing a light intensity distribution on the right surface (minus X-axis direction side surface) of each of the purification plates 14, 13, 12, 11. FIGS. 9B through 9E are gray-scale diagrams with respect to color diagrams.

In pattern 1, similarly to the arrangement shown in FIG. 3A, the light sources are disposed on the outside of the gaps to be defined between the purification plates 11 through 14. Further, two reflection plates are used for efficiently guiding light to be emitted from the light sources toward the purification plates 11 through 14. The configurations of the two reflection plates are expressed by y=0.0042x², y=0.0032x² in X-Z plane.

In the above arrangement, the light intensity is distributed on the right surface of each of the purification plates 11 through 14, as shown in FIGS. 9B through 9E. The light intensities on the right surfaces of the purification plates 14, 13, 12, 11 are respectively 0.50 watt, 0.42 watt, 0.27 watt, 0.12 watt; and the sum of these light intensities is 1.31 watts.

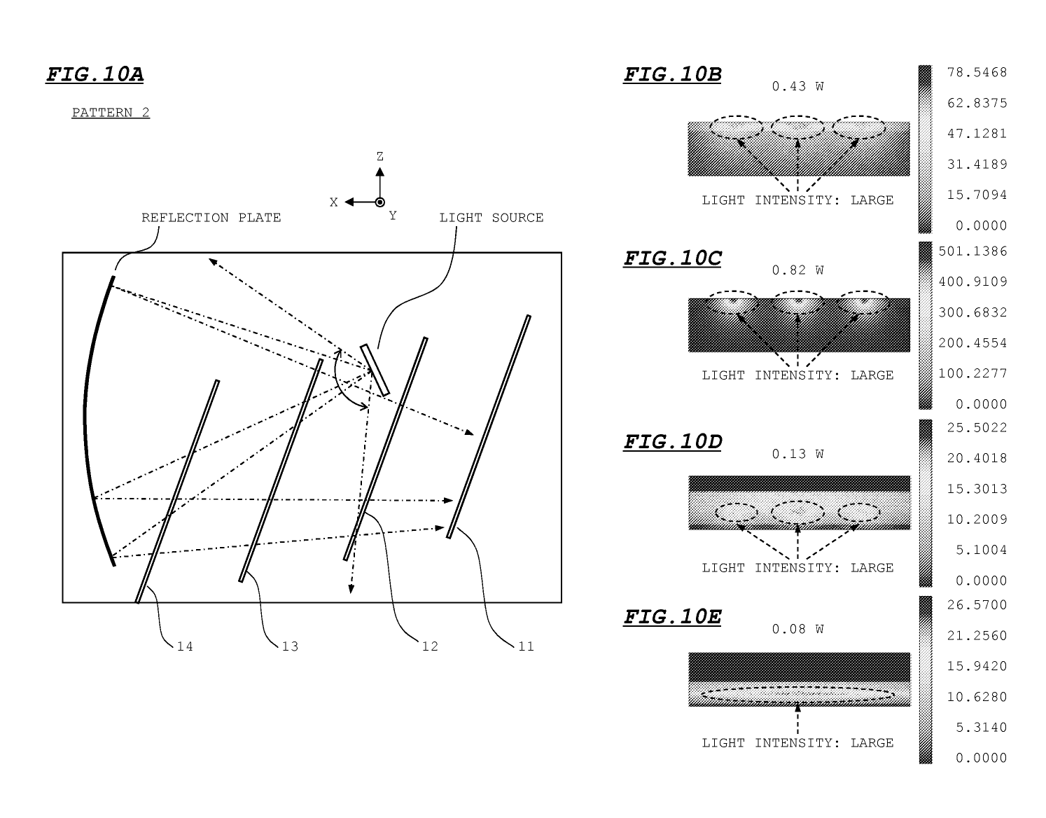

FIG. 10A is a schematic diagram showing the positions of light sources and a reflection plate in pattern 2. FIGS. 10B through 10E are simulation results respectively showing a light intensity distribution on the right surface (minus X-axis direction side surface) of each of the purification plates 14, 13, 12, 11.

In pattern 2, the light sources are disposed in the gap to be defined between the purification plates 12 and 13. Further, one reflection plate is used for efficiently guiding light to be emitted from the light sources toward the purification plates 11 through 14. The configuration of the reflection plate is expressed by y=0.005x² in X-Z plane.

In this arrangement, the light intensity is distributed on the right surface of each of the purification plates 11 through 14, as shown in FIGS. 10B through 10E. The light intensities on the right surfaces of the purification plates 14, 13, 12, 11 are respectively 0.43 watt, 0.82 watt, 0.13 watt, 0.08 watt; and the sum of these light intensities is 1.46 watts, which is larger than in pattern 1.

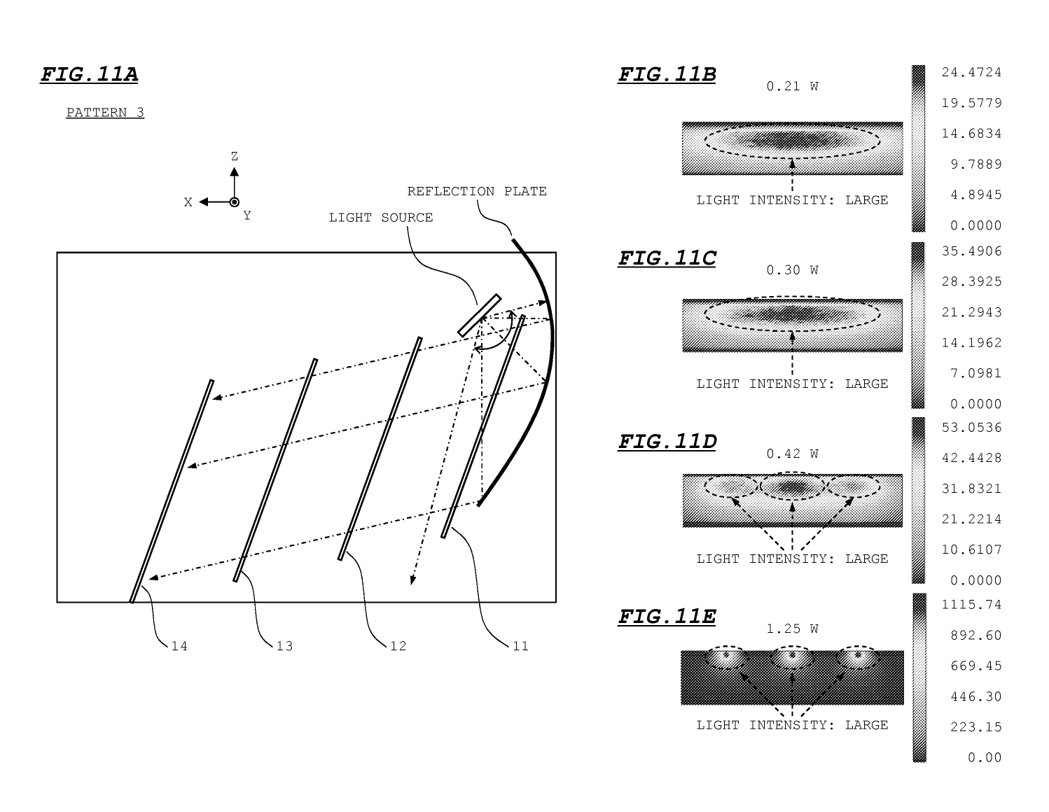

FIG. 11A is a schematic diagram showing the positions of light sources and a reflection plate in pattern 3. FIGS. 11B through 11E are simulation results respectively showing a light intensity distribution on the right surface (minus X-axis direction side surface) of each of the purification plates 14, 13, 12, 11.

In pattern 3, the light sources and the reflection plate are disposed in the same manner as the LEDs 21a and the reflection plate 31. The configuration of the reflection plate is expressed by y=0.013158x² in X-Z plane.

In the above arrangement, the light intensity is distributed on the right surface of each of the purification plates 11 through 14, as shown in FIGS. 11B through 11E. The light intensities on the right surfaces of the purification plates 14, 13, 12, 11 are respectively 0.21 watt, 0.30 watt, 0.42 watt, 1.25 watt; and the sum of these light intensities is 2.18 watts, which is larger than in pattern 2.

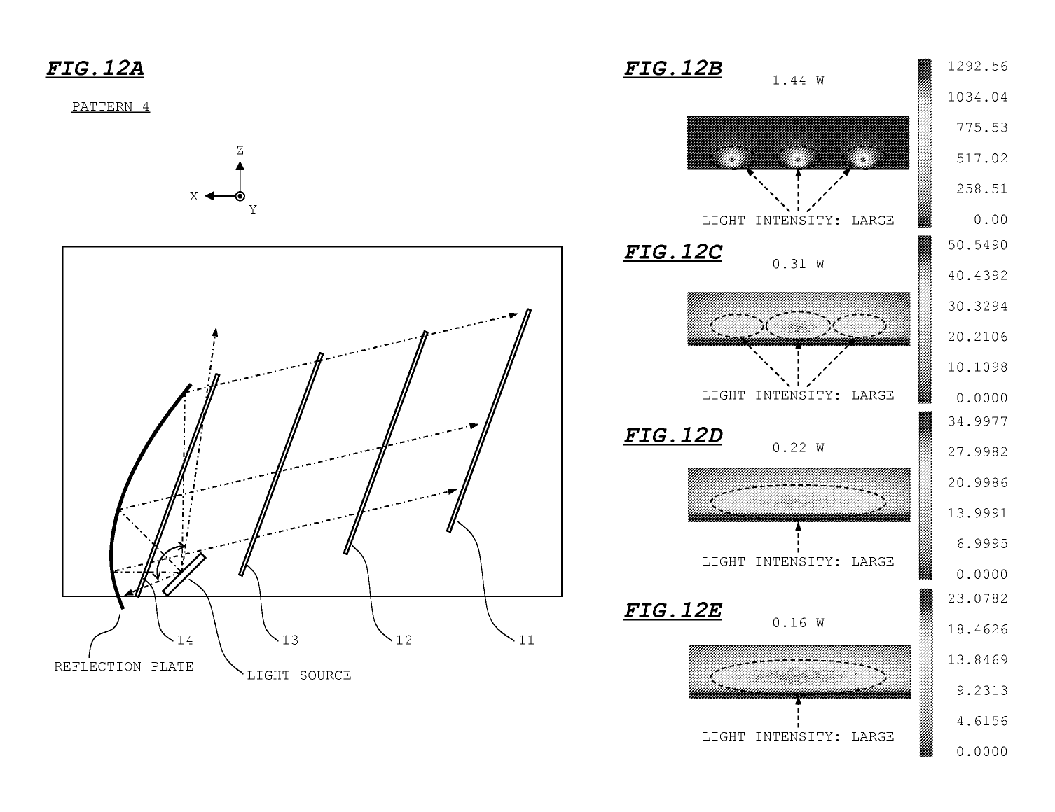

FIG. 12A is a schematic diagram showing the positions of light sources and a reflection plate in pattern 4. FIGS. 12B through 12E are simulation results respectively showing a light intensity distribution on the right surface (minus X-axis direction side surface) of each of the purification plates 14, 13, 12, 11.

In pattern 4, the light sources and the reflection plate are disposed in the same manner as the LEDs 22a and the reflection plate 32. The configuration of the reflection plate is expressed by y=0.013158x² in X-Z plane, which is the same as that of the reflection plate in pattern 3.

In the above arrangement, the light intensity is distributed on the right surface of each of the purification plates 11 through 14, as shown in FIGS. 12B through 12E. The light intensities on the right surfaces of the purification plates 14, 13, 12, 11 are respectively 1.44 watt, 0.31 watt, 0.22 watt, 0.16 watt; and the sum of these light intensities is 2.13 watts, which is substantially the same as in pattern 3.

As described above, the arrangement that light sources are disposed in one of the gaps to be defined between the purification plates 11 through 14, as shown by patterns 2 through 4, is advantageous in efficiently guiding the light onto the purification plates 11 through 14, as compared with the arrangement that light sources are disposed on the outside of the gaps to be defined between the purification plates 11 through 14, as shown by pattern 1. Further, the arrangement that light emitted from light sources is reflected on a reflection plate after transmittance through a purification plate only once, as shown by patterns 3, 4, is advantageous in efficiently guiding the light onto the purification plates 11 through 14, as compared with the arrangement that light sources are disposed in a gap to be defined between the purification plates 12 and 13, as shown by pattern 2.

As described above, with use of the purification unit 100 of the present example, the air drawn into the purification unit 100 through the vent 100a is purified by the photocatalyst films C13 of the purification plates 11 through 14, and is drawn out through the vent 100b. By performing the above operation, for instance, if the purification unit 100 is installed in a duct for flowing the air, the material to be purified that is contained in the air near an entrance of the duct is decomposed by the purification unit 100, and the purified air is fed to an exit of the duct. Thus, installing a duct provided with an entrance and an exit and loaded with the purification unit 100 in an air circulation path of a showcase for placing the goods in e.g. a supermarket or a convenience store, is advantageous in purifying the air within the showcase. In addition to the above, installing the purification unit 100 in a duct for circulating the air using a fan, such as an air circulation path in a refrigerator, is also beneficial.

Further, in the purification unit 100 of the present example, the LEDs 21a are disposed in the gap to be defined between the purification plates 11 and 12, and the LEDs 22a are disposed in the gap to be defined between the purification plates 13 and 14. This arrangement is advantageous in efficiently irradiating light onto the purification plates 11 through 14, as compared with the arrangement that the LEDs 21a, 22a are disposed on the outside of the purification plates 11 through 14, as shown in FIG. 3A and FIG. 9A. Thus, a photocatalytic reaction is efficiently caused on the purification plates 11 through 14. In the present example, light from the LEDs 21a, 22a can be efficiently irradiated onto the purification plates 11 through 14, as compared with the arrangement that the LEDs 21a, 22a are disposed in the gap to be defined between the purification plates 12 and 13, as shown in FIG. 10A.

Further, in the purification unit 100 of the present example, the LEDs 21a, 22a are respectively disposed in the gap to be defined between the purification plates 11 and 12, and in the gap to be defined between the purification plates 13 and 14. Further, these gaps serve as a channel along which the streams of air drawn into the purification unit 100 are allowed to flow. With this arrangement, since the LEDs 21a, 22a are cooled by the air, it is possible to suppress a temperature rise of the LEDs 21a, 22a. Thus, the above arrangement allows the LEDs 21a, 22a to stably emit light of a certain wavelength, and avoids a high-temperature condition of the purification unit 100.

Further, in the purification unit 100 of the present example, streams of air are allowed to flow in the purification unit 100 in the manner as shown in FIG. 8. Specifically, the direction of streams of air drawn in through the vent 100a is different from the direction of streams of air in the gaps to be defined between the purification plates 11 through 14. In this arrangement, the air is likely to stagnate within the purification unit 100, as compared with the arrangement that streams of air are allowed to flow in a spreading direction (left and right directions in FIG. 3B) of the gaps between the four purification plates 10, as shown in FIG. 3B. This allows the material to be purified that is contained in the air drawn in through the vent 100a to stagnate and easily adhere to the purification plates 11 through 14, which is advantageous in promoting the purification action, as compared with the arrangement as shown in FIG. 3B.

Further, in the purification unit 100 of the present example, streams of external air drawn in through the vent 100a are gradually blocked by the purification plates 11 through 14, and are completely blocked by the purification plate 14. With this arrangement, the streams of external air drawn in through the vent 100a are substantially uniformly distributed with respect to the purification plates 11 through 14. This is further advantageous in efficiently causing a photocatalytic reaction on the purification plates 11 through 14.

Further, in the purification unit 100 of the present example, the LEDs 21a, 22a are respectively disposed in the gap to be defined between the purification plates 11 and 12, and in the gap to be defined between the purification plates 13 and 14. This arrangement reduces the X-axis directional dimension of the purification unit 100, as compared with the arrangement that the light sources are disposed on the outside of the purification plates 11 through 14, as shown in FIG. 3B and FIG. 9A. Thus, it is possible to miniaturize the purification unit 100.

Further, in the purification unit 100 of the present example, the photocatalyst films C13 of the purification plates 11 through 14 are configured to cause a photocatalytic reaction by blue light of 405 nm wavelength. Generally, the manufacturing cost of LEDs, semiconductor laser light sources, and the like is increased, as the emission wavelength is shortened; and the manufacturing cost is reduced, as the emission wavelength approaches a visible light band. Since the manufacturing cost of the LEDs 21a, 22a in the purification unit 100 of the present example can be reduced, the cost relating to the purification unit 100 can be reduced. It is effective to use a material having a property that a photocatalytic reaction occurs specifically at light of 405 nm wavelength, as the material for the photocatalyst film C13. Specifically, a material containing TiO₂ loaded one of or a combination of Fe, Cu and Pt, and a material containing WO₃ loaded one of or a combination of Fe, Cu and Pt are suitable. These materials can effectively use visible light of 405 nm wavelength, as compared with a material containing rutile-type titanium oxide or the like.

### Example of Deodorizing Device

In the present example, a control circuit 170 corresponds to a controller in the claims. The description regarding the correspondence between the claims and the present example is merely an example, and the claims are not limited by the description of the present example.

The following is an example, wherein the aforementioned purification unit 100 is incorporated in a deodorizing device.

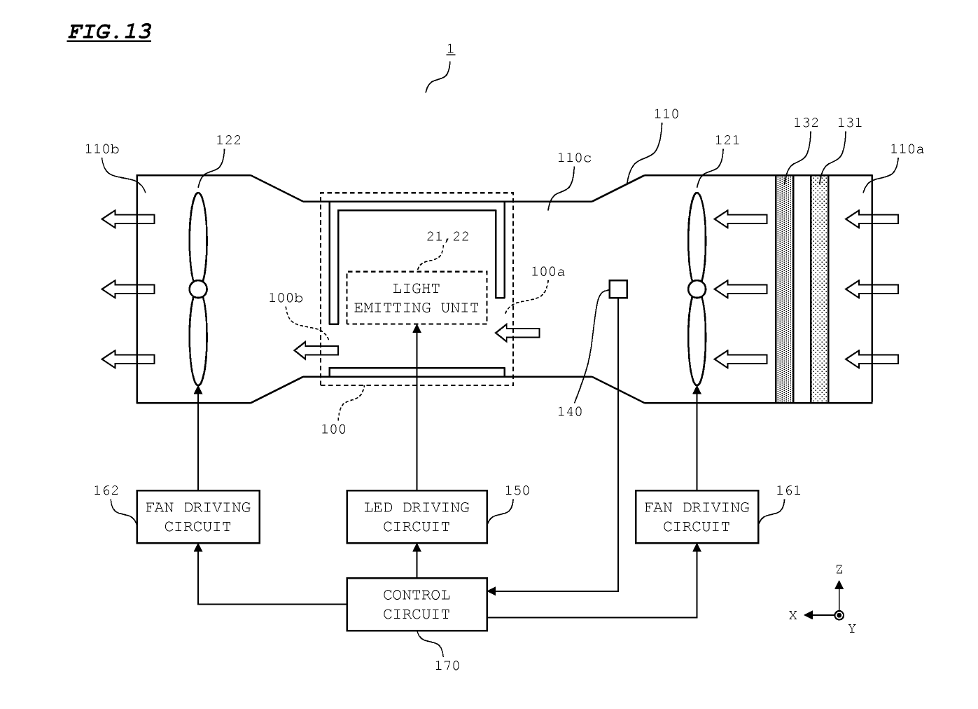

FIG. 13 is a diagram showing an arrangement of a deodorizing device 1.

The deodorizing device 1 is provided with a purification unit 100, an air feeding path 110, fans 121, 122, filters 131, 132, an odor sensor 140, an LED driving circuit 150, fan driving circuits 161, 162, and a control circuit 170.

The air feeding path 110 is formed into a hollow tubular member, and is so configured that the air is allowed to flow in plus X-axis direction. An entrance and an exit of the air feeding path 110 are respectively formed with an air intake port 110a and an air exhaust port 110b. Further, a purification region 110c for disposing the purification unit 100 is defined near the center of the air feeding path 110.

The purification unit 100 is the aforementioned purification unit 100. The vent 100a of the purification unit 100 faces toward the air intake port 110a, and the vent 100b of the purification unit 100 faces toward the air exhaust port 110b. In FIG. 13, the light emitting units 21, 22 are conceptually illustrated inside the purification unit 100, and the illustration of the other members of the purification unit 100 is omitted.

The fans 121, 122 cause the air to flow from the air intake port 110a toward the air exhaust port 110b. By performing the above operation, the air in the vicinity of the air intake port 110a is drawn in through the air intake port 110a by the fan 121, passes through the purification region 110c, and is drawn out through the air exhaust port 110b by the fan 122.

The filter 131 removes large dust particles contained in the air that is drawn in through the air intake port 110a, and the filter 132 removes small dust particles contained in the air that is drawn out from the filter 131 side. The odor sensor 140 detects an odor component contained in the air that is fed out toward the purification region 110c by the fan 121. A detection signal from the odor sensor 140 is outputted to the control circuit 170.

The LED driving circuit 150 drives the LEDs 21a, 22a disposed in the light emitting units 21, 22 in accordance with an instruction from the control circuit 170. The fan driving circuits 161, 162 respectively drive the fans 121, 122 in accordance with an instruction from the control circuit 170. The control circuit 170 controls the LED driving circuit 150, and the fan driving circuits 161, 162, based on an output signal from the odor sensor 140. For instance, in the case where a detection signal from the odor sensor 140 exceeds a predetermined value, the control circuit 170 controls the LED driving circuit 150 to increase the emission power of the LEDs 21a, 22a.

In the deodorizing device 1 thus constructed, dusts in the air drawn in through the air intake port 110a are removed by the filters 131, 132 by driving the fan 121, and then, the air after the dust removal is fed to the purification region 110c. The air fed to the purification region 110c is drawn into the purification unit 100, and as described above, the material to be purified is decomposed on the purification plates 11 through 14 in the purification unit 100.

The air that has been purified in the purification unit 100 is drawn out of the purification unit 100 through the vent 100b, and is fed toward the fan 122. The air in the purification region 110c is fed toward the air exhaust port 110b, and is fed out of the purification unit 100 through the air exhaust port 110b by driving the fans 121, 122. In this way, the air in the vicinity of the deodorizing device 1 is purified.

The embodiment of the invention has been described as above. The invention is not limited to the foregoing embodiment, and the embodiment of the invention may be changed or modified in various ways other than the above.

For instance, in the purification plates 11 through 14 of the example, the photocatalyst film C13 for causing a photocatalytic reaction by visible light of 405 nm wavelength is used. Alternatively, it may be possible to use a photocatalyst film having such a property as to cause a photocatalytic reaction by ultraviolet light (e.g. light of 375 nm wavelength).

Further, in the purification plates 11 through 14 of the example, as shown in FIG. 1D, the photocatalyst film C13 is formed to have a certain thickness. Alternatively, the photocatalyst film may have a varied thickness in X-axis direction or in Y-axis direction in FIG. 1D. In the modification, in FIG. 6, the purification plates 11 through 14 are disposed at such positions that an end of a photocatalyst film C13 having a larger thickness, out of the purification plates 11 through 14, is located at a lower position. The modification is advantageous in enhancing the purification performance of the purification unit 100, because the air containing a larger amount of a material to be purified is likely to be adhered to the end of the photocatalyst film C13 having a larger thickness.

Further, in the purification plates 11 through 14 of the example, as shown in FIG. 1D, the transparent film C12, the photocatalyst film C13, and the adsorption film C14 are laminated on both of the upper surface side and the lower surface side of the substrate C11. Alternatively, as shown in FIG. 1A, the transparent film C12, the photocatalyst film C13, and the adsorption film C14 may be laminated on one of the upper surface side and the lower surface side of the substrate C11.

Further, in the example, the LEDs 21a, 22a are used as light sources for causing a photocatalytic reaction. Alternatively, a semiconductor laser may be used, in place of the LEDs 21a, 22a. The semiconductor laser is a coherent light source, and is effective with respect to a specific crystal surface.

Further, in the example, the curved surface configuration of the reflection plates 31, 32 is expressed by a parabolic curve in X-Z plane. Alternatively, the curved surface configuration of the reflection plates 31, 32 may be expressed by other curve such as an elliptical curve. Further alternatively, the curved surface configurations of the reflection plates 31, 32 may be respectively expressed by different curves from each other in X-Z plane. In the modification, the incident angle of light reflected on the reflection plates 31, 32 with respect to the purification plates 11 through 14 is set to such a range as to suppress the ratio (light loss ratio) of light to be reflected on the purification plates 11 through 14, as well as in the example.

Further, in the example, four purification plates 11 through 14 are used. The number of the purification plates is not limited to the above, but may be a plural number other than four. In the case where five or more purification plates are used, the light emitting units 21, 22 may be disposed at such a position that light irradiation efficiency is highest with respect to all the purification plates, for instance, at a position between the second purification plate and the third purification plate with respect to the most upstream purification plate. Further, the position where light irradiation efficiency is highest with respect to all the purification plates may change depending on the interval between the purification plates or the emission powers of the light emitting units 21, 22. In view of the above, it is desirable to determine in which gap between the purification plates, the light emitting units 21, 22 should be disposed, depending on the number of the purification plates, the interval between the purification plates or the emission powers of the light emitting units 21, 22.

The embodiment of the invention may be changed or modified in various ways as necessary, as far as such changes and modifications do not depart from the scope of the claims of the invention hereinafter defined.

What is claimed is:

1. A purification unit for purifying air by a photocatalytic reaction, comprising:
a first light source which emits light;
a first reflection plate which reflects the light emitted from the first light source; and
a plurality of purification plates which cause the photocatalytic reaction by irradiation of the light emitted from the first light source, wherein
at least one of the purification plates is disposed between the first reflection plate and the first light source, and
the light emitted from the first light source is transmitted through the purification plate disposed between the first reflection plate and the first light source, and then is reflected on the first reflection plate for irradiation onto the purification plates.

2. The purification unit according to claim 1, wherein
one of the purification plates is disposed between the first reflection plate and the first light source.

3. The purification unit according to claim 1, further comprising:
a second reflection plate disposed on a side opposite to the first reflection plate with respect to the purification plates; and
a second light source which emits light of a same wavelength as a wavelength of the light to be emitted from the first light source, wherein
at least one of the purification plates is disposed between the second reflection plate and the second light source, and
the light emitted from the second light source is transmitted through the purification plate disposed between the second reflection plate and the second light source, and then is reflected on the second reflection plate for irradiation onto the purification plates.

4. The purification unit according to claim 1, wherein
a channel in the purification unit is configured in such a manner that a direction of streams of air in a gap to be defined between the purification plates is different from a direction of streams of air to be drawn into the purification unit.

5. The purification unit according to claim 4, wherein
the purification plates are disposed at such positions that the streams of air drawn into the purification unit through an air inlet of the purification unit are gradually blocked, and
the purification plate located at a position farthest from the air inlet completely blocks the streams of air drawn into the purification unit through the air inlet.

6. A deodorizing device, comprising:
a purification unit which purifies air by a photocatalytic reaction;
a fan which causes the air to flow into the deodorizing device; and
a controller which controls the fan and the purification unit,
the purification unit including:
a first light source which emits light;
a first reflection plate which reflects the light emitted from the first light source; and
a plurality of purification plates which cause the photocatalytic reaction by irradiation of the light emitted from the first light source, wherein
at least one of the purification plates is disposed between the first reflection plate and the first light source, and
the light emitted from the first light source is transmitted through the purification plate disposed between the first reflection plate and the first light source, and then is reflected on the first reflection plate for irradiation onto the purification plates.

7. The deodorizing device according to claim 6, wherein
one of the purification plates is disposed between the first reflection plate and the first light source.

8. The deodorizing device according to claim 6, further comprising:
a second reflection plate disposed on a side opposite to the first reflection plate with respect to the purification plates; and
a second light source which emits light of a same wavelength as a wavelength of the light to be emitted from the first light source, wherein
at least one of the purification plates is disposed between the second reflection plate and the second light source, and
the light emitted from the second light source is transmitted through the purification plate disposed between the second reflection plate and the second light source, and then is reflected on the second reflection plate for irradiation onto the purification plates.

9. The deodorizing device according to claim 6, wherein
a channel in the purification unit is configured in such a manner that a direction of streams of air in a gap to be defined between the purification plates is different from a direction of streams of air to be drawn into the purification unit.

10. The deodorizing device according to claim 9, wherein
the purification plates are disposed at such positions that the streams of air drawn into the purification unit through an air inlet of the purification unit are gradually blocked, and
the purification plate located at a position farthest from the air inlet completely blocks the streams of air drawn into the purification unit through the air inlet.
